(19) United States
(12) Patent Application Publication
(10) Pub. No.: US 2005/0283104 A1
(43) Pub. Date: Dec. 22, 2005

(54) Back therapy device

(76) Inventor: Kevin G. Abelbeck, Lincoln, NE (US)

Correspondence Address: Kevin G. Abelbeck, 7455 South 28th Street, Lincoln, NE, 68516, US

(21) Appl. No.: 10/872,180

(22) Filed: Jun. 18, 2004

(51) Int. Cl. A61F 5/00

(52) U.S. Cl. 602/32; 606/241; 128/845

(57) ABSTRACT

An inflatable bladder is used to articulate the spine of a user. A pelvis pad includes a support frame and a pliable cushion positioned thereon. The bladder is supported on the support frame and received by a void in the cushion. In the preferred embodiment the bladder includes two upper portions that extend away from the support frame toward the body of the user. The upper portions of the bladder, or two separate bladders, are cylindrical in shape when expanded. The void in the cushion is also cylindrical in shape and therefore acts to guide the expansion and contraction of the bladders or upper portions of the bladder. The upper portions are preferably positioned eleven inches apart and inflate at least two inches above the support frame. This proximity of the upper portions of the bladder fit the width of the right and left anterior superior illiac spine along the illiac crest of the pelvis. Expansion of the bladder applies a posterior force to the pelvis through the illiac crest and rotates the pelvis. This manipulates the lumbar spine to stretch the muscles of the lower back and increase the posterior disk spacing of the lumbar vertebrae, providing mild lumbar traction.

### FIELD OF THE INVENTION

The present invention generally relates to therapy devices. More specifically, the present invention relates to devices for relief of acute lower back pain.

### BACKGROUND OF THE INVENTION

Low back pain is the fifth-leading cause of trips to the doctor in the United States, according to the Mayo Clinic (Mayo Clinic Health Information, Jun. 3, 2004). The same source notes that back injuries are the leading cause of work-related disability. The National Institute of Neurological Disorders and Stroke reports that Americans spend at least $50 billion each year on low back pain. It is also estimated that approximately 80% of all Americans will encounter a bout with low back pain at some time in their lives (Mayo Clinic, 2004). When lost wages and work time are also considered, the loss to the American society is staggering.

Traction devices have been used for many years with some success. The concept is to apply a tension load to the spine. In theory, by pulling the spine longitudinally, a bulging or otherwise prolapsed disk between the vertebrae would be pulled in and away from the nerve cord located just posterior to the spine. Due to the lordosis or lumbar curve of the spine, applying a random load can cause the muscles to tighten and increase the arch or curve. This may increase the displacement of the disk bulge toward the nerve cord, exacerbating the problem. Traction must be controlled not only by the level of tension but the support of the body. Proper manipulation of the spine and pelvis can in itself provide a controlled traction to the spine.

### SUMMARY OF THE INVENTION

In one aspect, the invention is a back therapy device, which features a pelvis pad with a pliable portion and at least one cavity preferably being substantially cylindrical in shape. In the preferred embodiment there are two cavities positioned between nine and thirteen inches apart and optimally eleven inches apart. A support frame is provided, being comprised substantially of a channel with a pair of sloping legs and a hollow underside. The support frame is disposed substantially under the pliable portion, which may be a foam rubber pad (such as closed cell polyurethane). At least one collapsible bladder is supported by the support frame and received by the cavity. The bladder includes two upper portions that expand away from the support frame by at least one inch or in the preferred embodiment by at least two inches. These upper portions may be part of the same continuous bladder or they may be two separate bladders each with their own upper portion. The upper portions of the bladder, or the bladders themselves, are preferably substantially cylindrical in shape and comprised of a pliable material such as polyvinyl chloride (PVC) vinyl.

A pump is provided in fluid communication with the bladder, whereby actuation of the pump enables expansion of the bladder, thus providing vertical displacement of the upper portions of the bladder relative to the support frame. The pump can be an electrically powered pump such as an air pump or other fluid power pump, or a hand pump. The electrical pump may be operated by use of a switch remotely positioned from the pump, such as positioned adjacent to a face pad, which may also be included in the system.

The face pad may be comprised of a substantially U-shaped face pad with an open center section and an upper connecting section. The open center section may include an expanded top portion, adjacent to the connecting section, whereby when a user's face is positioned thereon, the expanded top portion provides room lateral to the eyes of the user for increased comfort. Another addition to increase comfort includes providing a void near the base of the connecting section, thereby providing an air breathing passage for the user.

The system my also include a substantially flat chest mat connected to the pelvis pad and to the face pad. A pliable substantially cylindrical shin pad may also be added. The shin pad provides support for the lower legs of a user in a prone position when the midsection of the user is positioned on the pelvis pad.

In another aspect, the invention includes a method of manipulating the spine of the user providing a structure as previously noted. The method includes positioning a user in a prone position with the user's illiac crest over the bladder. The user actuates the pump, thereby expanding the bladder, displacing it away from the frame. In doing so, this provides posterior rotation of the user's pelvis by a force applied to the illiac crest from the expanding bladder.

### BRIEF DESCRIPTION OF THE DRAWINGS

The foregoing and other objects of this invention, the various features thereof, as well as the invention itself, may be more fully understood from the following description, when read together with the accompanying drawings, described:

FIG. 1a is a side view of a person lying in a prone position on a flat surface.

FIG. 1b is a side view of a person lying in a prone position with the pelvis on an inflated pelvis pad, the head supported on a face pad and the lower legs supported on a leg roll pad, the device in combination produced in accordance with the present invention.

FIG. 2 is an isometric view of a version of a back therapy device including a pelvis pad, face pad and a leg roll pad, the device in combination produced in accordance with the present invention.

FIG. 3 is an isometric view of a version of a back therapy device including a pelvis pad with inflated bladders and a face pad, the device produced in accordance with the present invention.

FIG. 4a is a schematic of the electrical and pneumatic systems of one version of the device with a common bladder, the device produced in accordance with the present invention.

FIG. 4b is a portion of the schematic similar to that shown in FIG. 4a, showing an alternative valve to enable two unique bladders, the device produced in accordance with the present invention.

FIG. 5 is an exploded isometric view of a version of a pelvis pad of a back therapy device, the device produced in accordance with the present invention.

FIG. 6 is an isometric view of a version of a back therapy device including a pelvis pad with one inflated bladder and one deflated bladder and a face pad, the cushion of the pelvis pad shown displaced, the device produced in accordance with the present invention.

FIG. 7 is an isometric view of a version of a back therapy device including a pelvis pad with deflated bladders and a face pad, the cushion of the pelvis pad shown displaced, the device produced in accordance with the present invention.

FIG. 8 is an isometric view of a version of a back therapy device including a pelvis pad with deflated bladders and a face pad, the cushion of the pelvis pad shown vertically displaced and the electric pump replaced with a hand pump, the device produced in accordance with the present invention.

For the most part, and as will be apparent when referring to the figures, when an item is used unchanged in more than one figure, it is identified by the same alphanumeric reference indicator in all figures.

### DETAILED DESCRIPTION OF THE PREFERRED EMBODIMENT

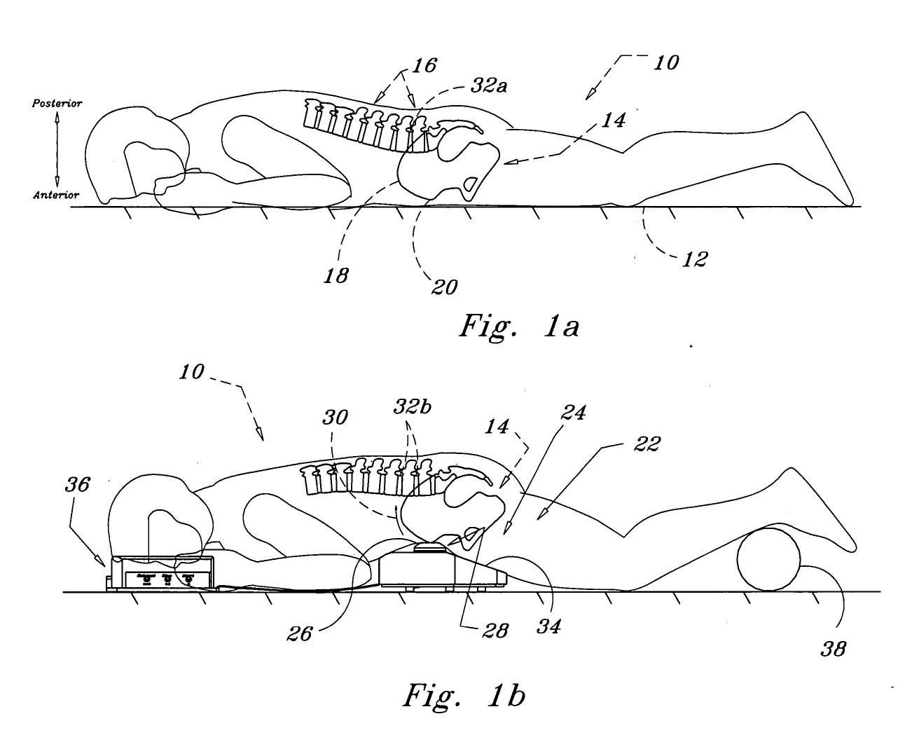

The present invention is a back therapy device. The general function of the device is shown in FIGS. 1a and 1b. In FIG. 1a, a person 10 is shown lying in a face down in a prone position on a flat surface 12. The pelvis 14 of the person 10 is illustrated in its natural position with the lumbar spine 16 including its natural curve or lordosis. The front sweeping portion of the pelvis is the illiac crest 18. Toward the anterior (in this view the lower) portion a bony protrusion exists. This protrusion is the anterior, superior illiac spine 20. The pelvis is supported by the long bones of the upper legs or the femur (not shown).

The same body is shown while using the device 22 in FIG. 1b. The invention may include some peripheral components, as will be disclosed in detail, but the primary component is the pelvis pad 24. This is positioned under the pelvis 14 of the user 10 with an upper portion 26 of a bladder 28 positioned under the illiac crest of the user. Optimally the anterior, superior illiac spine is targeted. The bladder 28 is shown here to be inflated and thereby rotating the pelvis 14 toward the back or posterior side of the user 10 as indicated by the arrow 30. In doing so, the lordosis of the lumbar spine is reduced to be substantially flat. Upon closer examination of the distances between the posterior aspects of the lumbar vertebrae, it can be seen that they are dramatically increased from that in the flat condition 32a to that in the posterior pelvic tilt position 32b. The nerve cord runs posterior to the disks between these vertebrae and therefore any bulge in these disks will likely protrude toward the nerve cord. This spinal manipulation not only stretches the muscles of the lower back, but also acts to mildly pull the posterior aspects of these vertebrae apart (32a to 32b). This provides a mild traction to the lumbar spine 16.

Other aspects of the invention 22 include the sloped section 34 of the pelvis pad 24 to more comfortably support the legs of a user 10. Additionally, a face pad 36 is positioned in front of the pelvis pad 24 and used to comfortably support the head of the user 10 in a face down or prone position. A shin pad 38 is also provided to allow the user 10 to lie in this position with the knees slightly bent allowing the biceps femoris, semitendinosus and other muscles of the hamstrings to relax.

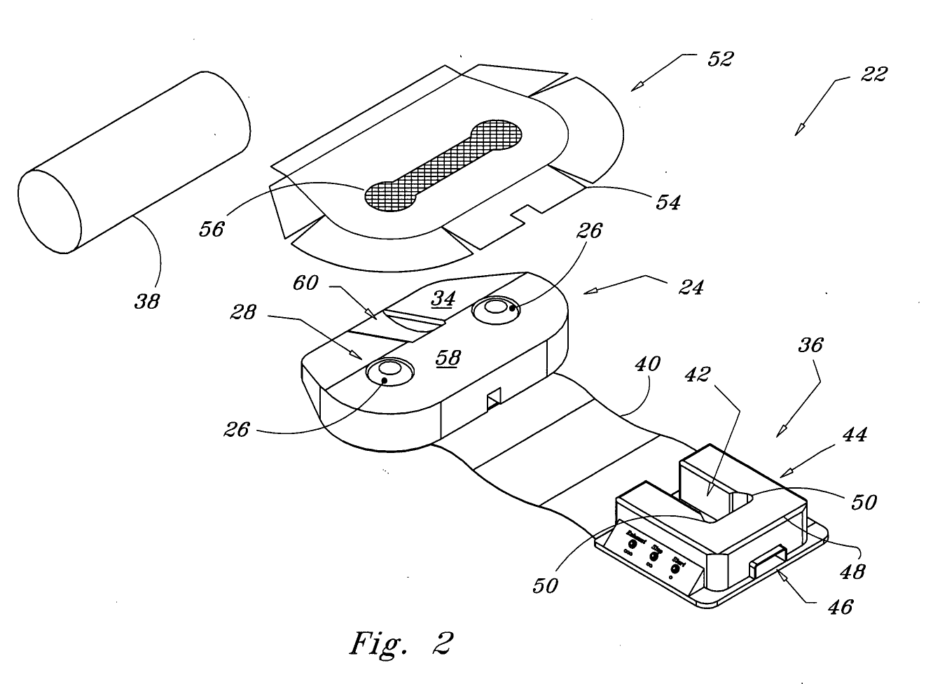

The device 22 is shown alone in FIG. 2. The face pad 36 is connected to the pelvis pad 24 by a chest mat 40. The chest mat 40 is not a required element of the invention, but it functions to tether the face pad 36 to the pelvis pad 24 as well as offering a clean environment as opposed to the floor, where the device 22 would typically be positioned when in use. The user's face is positioned within the open center section 42 of the substantially U-shaped pad 44 of the face pad 36. Therefore movement of air for breathing will partially come from under the user's chest, which would be on top of the chest mat 40. Since the mat 40 is not walked on as much as a typically floor, it is easier to prevent contaminants from being sucked into the respiratory system of the user. Additional airflow is provided by the air tunnel 46, which is a void near the base of the connecting section 48 of the U-shaped face pad 44.

When positioned for a prolonged period on a typical face down pad, the user's eye sockets can experience pressure from the pad 44 and cause discomfort. To solve this problem, the open center section 42 includes an expanded top portion 50 adjacent to the connecting portion 48. This expanded portion 50 on each side allows for the forehead and the side of the face of the user to support the weight of the head, avoiding contact with the area around the eyes. This reduces pressure on the zygomaticofacial nerve and middle temporal vein next to the eye.

The pelvis pad 24 is the primary functional unit of the device 22. It is shown here with the pad cover 52, typically a vinyl or leather material, removed to better show the components of the pelvis pad 24. The pad cover 52 is in a flat pattern to illustrate what it is and how it might be manufactured. The cuts 54 would be sewn together to provide a “cap” that fits over the pelvis pad structure and fastened to the base of the pelvis pad 24. An elastic section 56 is shown as a mesh element secured into the pad cover 52. This is done to allow for the vertical displacement of the upper portions 26 of the bladder 28. The elastic properties of the elastic section 56 could alternatively be incorporated into the material of the pad cover 52, making it a homogenous material that is capable of deformation with memory. This would enable the bladder 28 to expand and deflate repeatedly over time without a structural failure of the pad cover 52. The alternative is to sew in an elastic section 56 comprised of a specialized material with good elastic properties that will allow for the deformation. This is the embodiment illustrated here.

The pelvis pad 24 further includes the bladder 28 with two upper portions 26. This will be shown in greater detail later in the disclosure. The upper portions 26 are the parts of the bladder that expand upward toward the user to apply a force to manipulate the pelvis. In the preferred embodiment, this can be accomplished by two separate bladders, each with an upper portion, or by one bladder with two upper portions. A single bladder with one upper portion can also be used which would manipulate one side of the pelvis at a time. Though this could be made functional, it is considered by the inventor that the preferred embodiment includes two upper portions that contact each side of the user's illiac crest simultaneously or alternatively but without the need for the user to move their body from one side to the other. As such, the disclosure only shows two upper portions 26, though it is understood that one could be made to work.

The pad 24 includes a flat section 58 and a sloped section 34. The flat section 58 provides a general support platform for the user's midsection. The flat section 58 can be manufactured of a semi-rigid material, but it is considered desirable for comfort that the flat section 58 and the sloped section 34 be manufactured of a pliable material such as foam rubber. A molded piece of closed cell polyurethane that incorporates both the flat 58 and sloped 34 sections is preferred. As noted prior, the sloped section 34 is provided to more ergonomically support the upper legs of the user. For male users, a center cutaway 60 is provided to allow extra room for the groin.

A shin pad 38 is provided to offer support for the lower legs of the user. This can take a variety of forms and is shown here as a cylindrical block. The specific shape is not critical to the invention but the cylindrical form is considered optimal in that while the user is positioned face down, the user can roll the shin pad 38 closer to the feet or further from the feet with minimal effort in order that a comfortable position can be attained. As with a portion of the pelvis pad 24, a foam rubber material is preferred.

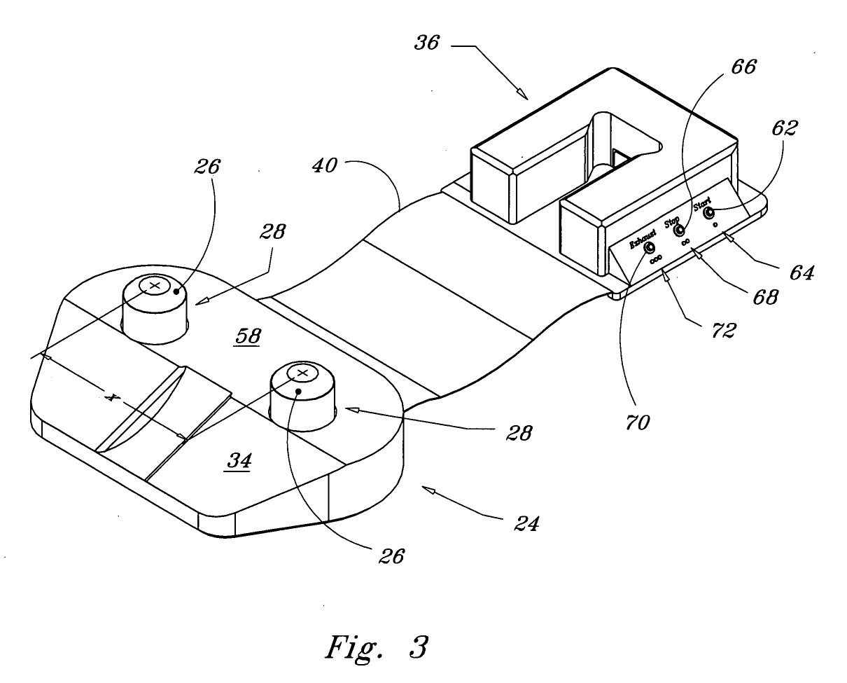

Greater detail of the pelvis pad 24 and face pad 36 is shown in FIG. 3. The pelvis pad 24 is changed in that the upper portions 26 of the bladder 28 have been expanded to extend up beyond the top of the flat surface 58 of the pad 24. Here it can be seen that the upper portions 26 are substantially cylindrical in shape. Again, this can be one continuous bladder with two upper portions 26 or two unique bladders 28. The dimension “x” designates the relative location of these upper portions 26. The upper portions 26 are intended to support each of the two anterior aspects of the superior illiac spine on the right and left side of the pelvis of the user. The lateral spacing of these pelvic bones is between nine and thirteen inches apart, with an optimal positioning of eleven inches (x). The vertical displacement of these upper portions 26 is at least one inch and preferably at least two inches when not under load. The bladder 28 is supported by an internal frame, which will be later disclosed in more detail. The bladder 28 is preferably collapsible in that it can be deflated when air or other fluid is allowed to exit the bladder 28, thus allowing the upper portions 26 to retract back toward the frame located within the pelvis pad 24. A suitable material for the bladder 28 is polyvinyl chloride (PVC) vinyl. This is a pliable plastic material that is very durable and commonly used for inflatable products such as pool toys.

In this embodiment of the invention, a pump is housed within the pelvis pad 24. This is preferably an electrically powered air pump. This will be further disclosed in more detail. The control of this pump and the rest of the fluid power system are remotely operated by one or more switches. In this embodiment, three switches are used and located on the base of the face pad 36. The switches can be of any form to control the pump and other aspects of the fluid power controlling the bladder 28. A common form would be electric switches, but pneumatic switches would also work with an air actuated system. Given that electrical communication is more easily achieved between the face pad 36 and the pump and other controls in the pelvis pad 24, an electric system is preferred.

A start button 62 is a switch positioned near the face pad 36 and thereby easily accessible to the user in a face down position. A single raised dot 64 can be used as a sightless communication of this the proper button to initiate the action of the pump to expand the bladder 28. When an air, hydraulic or other fluid power pump is used to expand the bladder 28, a pressure switch is used to shut the pump off at a predetermined maximum pressure. In the event that the user desires to stop the pump prior to maximal expansion, a stop switch 66 is provided. As before, a sightless identifier is provided with two raised dots 68 next to this switch 66. Finally to release the air or other fluid from the bladder 28, an exhaust switch 70 is used. A raised three-dot group 72 is located next to the exhaust button 70.

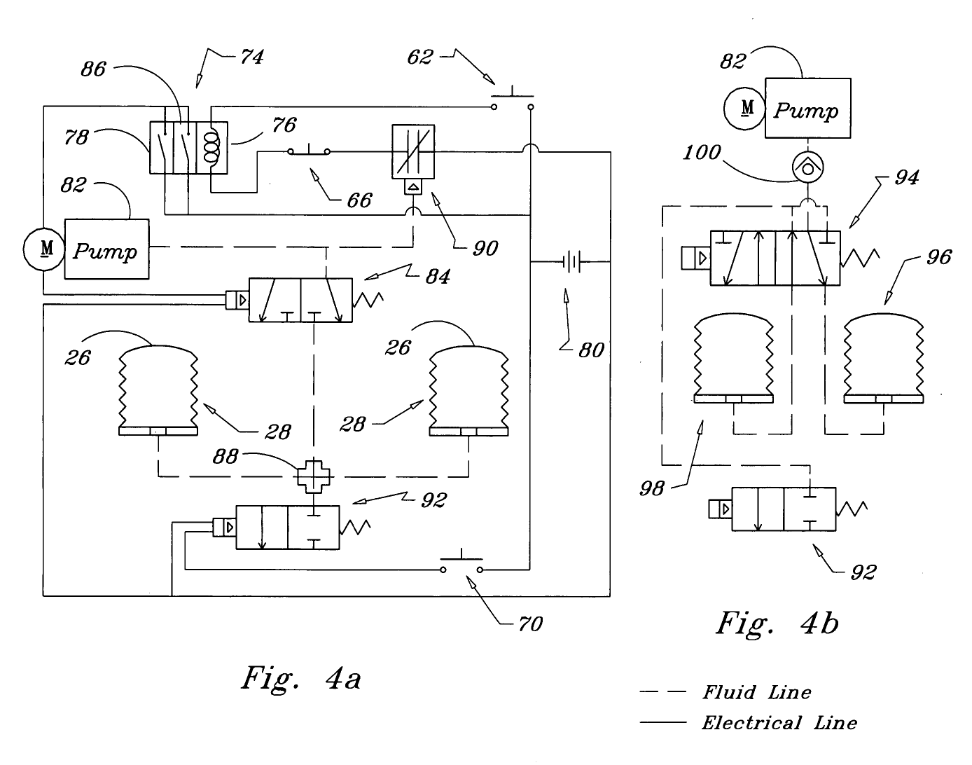

In this form of the invention, electric, spring return momentary switches are used. This system is detailed by a schematic in FIG. 4a. A current is provided to the circuit upon closing the start switch 62. This actuates a relay switch 74, such as a double pole double throw (DPDT) solenoid relay switch. Energizing the coil 76 of the relay switch 74 closes two normally open switches. The first relay switch 78 closes the electric circuit from a battery 80 to the motor of the pump 82. This circuit also provides voltage to energize the solenoid of a 3-way solenoid valve 84. The second relay switch 86 provides a closed circuit from the battery 80 to the coil 76, thus enabling the relay switches (78 & 86) to remain closed after the start switch 62 (normally open momentary switch) is released. The valve 84 provides fluid communication between the pump 82 and the bladder 28. In that the two separate structures 28 are connected by a common airline 88, the two-bag structure functions as one bladder 28 with two unique upper portions 26. Upon reaching maximum pressure, the normally closed pressure switch 90 opens or the stop switch 66 (normally closed momentary switch) is opened, thus interrupting the voltage to the coil 76 of the relay switch 74. This opens the relay switches (78 & 86) stopping the current flow to the motor of the pump 82 and returning the 3-way valve 84 to the relaxed position shown. This traps the fluid in the bladder 28.

To release the fluid from the bladder 28, the exhaust button 70 (normally open momentary switch) is actuated completing the circuit from the battery 80 to the solenoid of the 2-way solenoid valve 92. This provides an escape for the air or other fluid within the bladder 28. Upon release of the exhaust switch 70, the 2-way valve 92 returns to the relaxed locked position To release more fluid, the user depresses and holds the exhaust switch 70. Upon release of the exhaust switch 70, the valve automatically resets to hold fluid in the bladder 28.

A 4-way valve 94 could be substituted for the 3-way valve 84, thus separating and thereby providing a unique substantially cylindrical shaped first bladder 96 and a second bladder 98. This is shown in FIG. 4b. The pump 82 provides fluid flow through the check valve 100 and then to the 4-way valve 94. The 4-way valve 94 directs fluid flow to either the first bladder 96 (relaxed valve) or the second bladder 98 (energized valve). The exhaust of each bladder is controlled by the 2-way valve 92 and prevented from back flowing through the pump 82 by the check valve 100. By shuttling the 4-way valve 94 in either the stage of inflow from the pump 82 or exhaust through the 2-way valve 92, each of the first and second bladders (96 & 98 respectively) can be controlled independently. If a consistent inflow or exhaust is desired, the 4-way valve 94 can be actuated at a relatively high rate (over 2 Hz) to fill or exhaust both bladders with frequent small changes. This system would be especially valuable for electronically programmed sessions where one bladder is desired to expand or deflate independently of the second bladder.

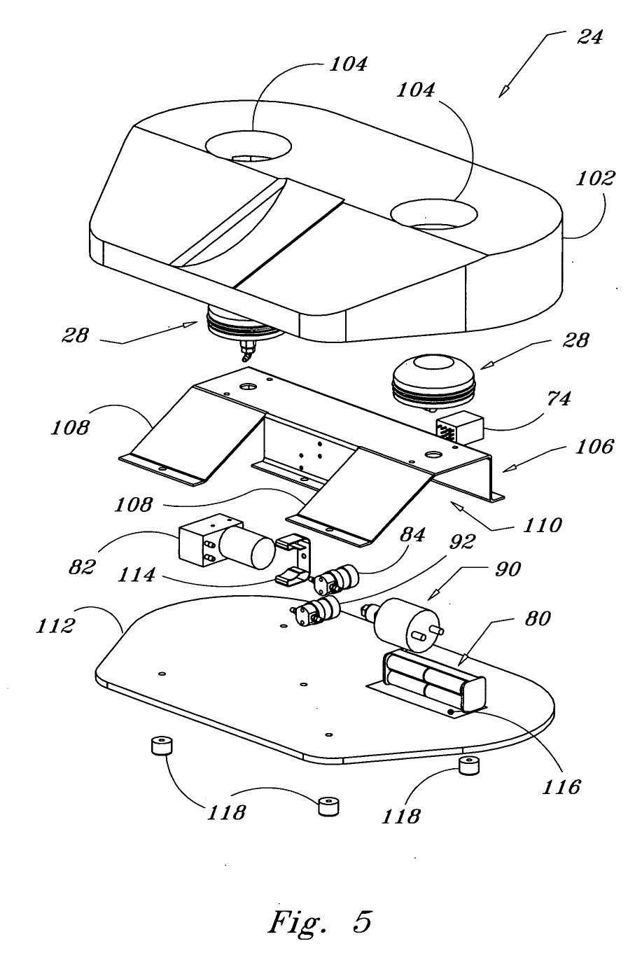

An exploded view of the pelvis pad 24 is shown in FIG. 5. As stated earlier the pad cover is not shown though it is understood that some type of covering may be used to provide a surface that is easily cleaned between uses. The pelvis pad 24 includes a pliable portion 102 (preferably manufactured of foam rubber) that includes two cylindrical shaped cavities 104. These cavities 104 act to guide the expansion and deflation of the bladders 28, which have the spacing relative to each other as previously noted. The bladders 28 (shown here in a deflated state) are supported by a support frame 106, which is comprised substantially of a channel with two sloping legs 108. The channel form of the support frame 106 allows for a substantially hollow underside 110. This hollow underside 110 with the base 112, together provides a structural container to house and support the drive and control components for the pelvis pad 24.

The bladders 28 are mounted to the top of the support frame 106, thus providing a secure base of support from which the bladders 28 can expand up through the voids 104 in the pliable portion 102. The support frame 106 can be part of the base 112, or as shown here to be mounted thereto. The relay switch 74, valves (84 & 92) and pressure switch 90 are all mounted to the support frame 106. The pump 82 may be mounted to the support frame 106 by way of the clip 114 shown here. The batteries 80 are mounted to the base 112, on a battery door 116, which is pivotally mounted to the base 112. This facilitates replacement of the batteries 80 when they run low. A series of feet 118 are provided to elevate the bottom of the base 112 off the floor or other supportive surface during use and storage.

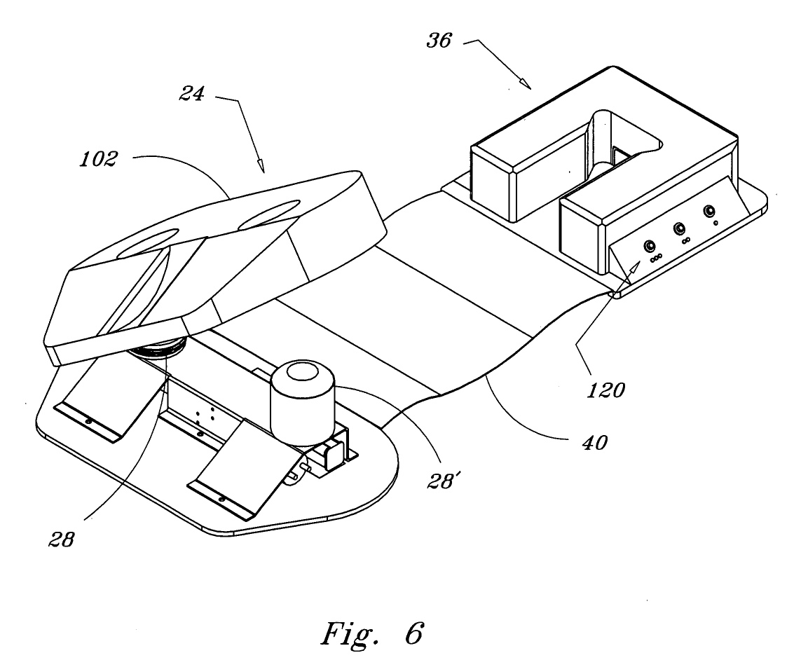

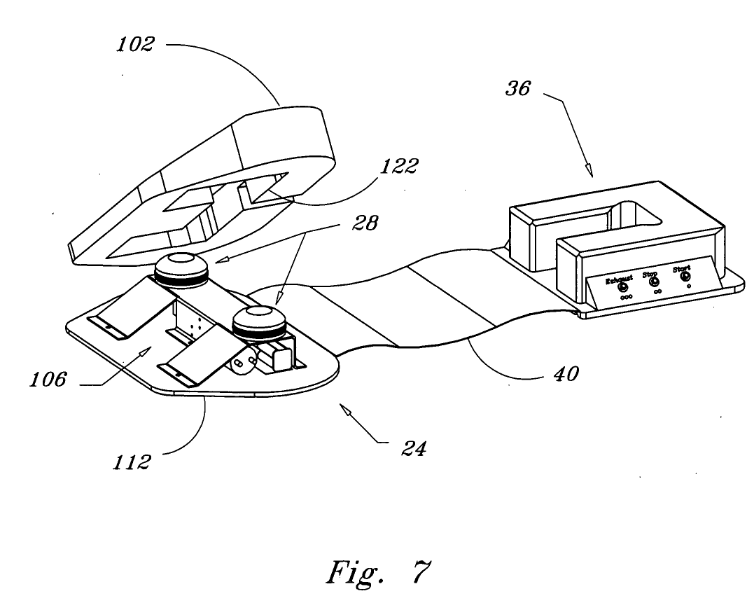

The pelvis pad 24 with the chest mat 40 and face pad 36 are shown in FIGS. 6 & 7 in slightly different views with the pliable portion 102 displaced. The pelvis pad 24 is assembled with the exception of the pliable portion 102. FIG. 6 shows the pelvis pad 24 with one bladder inflated 28′ and one deflated 28. This can be achieved by use of the system as previously disclosed in FIG. 4b. Though possible to use the afore disclosed start, stop, exhaust system, it is more likely this would be used with a programmable system utilizing a microprocessor to actuate the valves and pump in accordance with a predetermined protocol. An infinite number of protocols can be provided to the user to quickly or slowly inflate or deflate one or both bladders 28. In view of this use, a series of buttons 120 can still be used, each for a pre-programmed protocol, each potentially lasting several minutes. Fig, 7 shows a similar view with two bladders 28 that are both deflated. In both cases the bladders 28 are mounted to the support frame 106. The pliable portion 102 is revealed showing the under side. An additional void 122 is provided to receive the structure of the pelvis pad 24 that is mounted to the base 112.

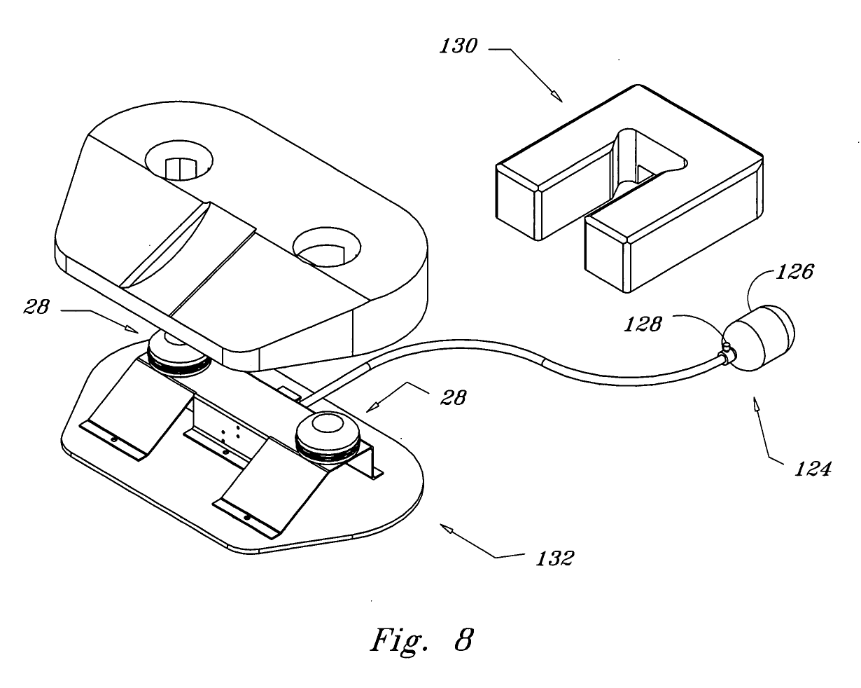

An alternate version of the invention is shown in FIG. 8. Here the electric pump has been replaced with a hand pump 124. This pump can be similar to the hand pump used on a mechanical blood pressure cuff or sphygmomanometer. It consists of a bulb 124 with a screw valve 128. When the valve 128 is closed a pumping action of the bulb 126 pumps air into the bladder 28. By unscrewing the valve 128, air is allowed to exit the system and deflate the bladder 28. In this version no batteries or other valves are needed. As such, the face pad 130 is simplified to eliminate the base support, which previously housed the switches. The chest mat has also been eliminated in this version in that there are no wires traveling between the simplified face pad 130 and the modified pelvis pad 132.

The invention as shown and described herein is the preferred embodiment of the invention as seen by the inventor. It is understood that an infinite number of variations of certain details are possible and therefore are inherently included in this disclosure.

1. A back therapy device comprising:
a pelvis pad with a pliable portion and at least one cavity therein;
a support frame disposed substantially under said pliable portion;
at least one bladder supported by said support frame and received by said at least one cavity; and
a pump in fluid communication with said at least one bladder, whereby actuation of said pump enables expansion of said at least one bladder providing vertical displacement of a portion of said at least one bladder relative to support frame.

2. A therapy device as in claim 1, wherein said pliable portion is comprised of a foam pad.

3. A therapy device as in claim 2, wherein said foam pad is manufactured from closed cell polyurethane.

4. A therapy device as in claim 1, wherein said at least one cavity is substantially cylindrical in shape.

5. A therapy device as in claim 1, wherein said at least one cavity is exactly two cavities positioned between nine and thirteen inches apart.

6. A therapy device as in claim 1, wherein said at least one cavity is exactly two cavities positioned eleven inches apart.

7. A therapy device as in claim 1, wherein said support frame is comprised substantially of a channel with a pair of sloping legs.

8. A therapy device as in claim 7, wherein said channel includes a substantially hollow underside.

9. A therapy device as in claim 1, wherein said at least one bladder is a collapsible bladder.

10. A therapy device as in claim 1, wherein said at least one bladder includes exactly two expandable upper portions, received by two cavities in said pliable portion, the expandable upper portions capable of displacement substantially away from said support frame.

11. A therapy device as in claim 10, wherein said exactly two expandable upper portions are positioned between nine and thirteen inches apart.

12. A therapy device as in claim 10, wherein said exactly two expandable upper portions are positioned eleven inches apart.

13. A therapy device as in claim 1, wherein said at least one bladder includes an upper portion that extends away from said support frame at least one inch when said at least one bladder is expanded.

14. A therapy device as in claim 1, wherein said at least one bladder includes an upper portion that extends away from said support frame at least two inches when said at least one bladder is expanded.

15. A therapy device as in claim 1, wherein said at least one bladder is comprised of two substantially cylindrical shaped bladders comprised of a pliable material.

16. A therapy device as in claim 15, wherein said pliable material is polyvinyl chloride vinyl.

17. A therapy device as in claim 1, wherein said pump is an electrically powered air pump.

18. A therapy device as in claim 1, wherein said pump is operated by use of a switch remotely positioned from said pump.

19. A therapy device as in claim 1, wherein said pump is hand actuated.

20. A therapy device as in claim 1, further comprising a substantially U-shaped face pad with an open center section and an upper connecting section.

21. A therapy device as in claim 20, wherein said open center section includes an expanded top portion, adjacent to said connecting section, whereby when a user's face is positioned thereon, said expanded top portion provides room lateral to the eyes of the user.

22. A therapy device as in claim 20, wherein said connecting section includes a void near the base thereof, thereby providing air passage to a user positioned therein.

23. A therapy device as in claim 20, wherein said pump is actuated by a switch located adjacent to said substantially U-shaped face pad.

24. A therapy device as in claim 1, further comprising a substantially flat chest mat connected to said pelvis pad and to a face pad.

25. A therapy device as in claim 1, further comprising a pliable shin pad, whereby said shin pad provides support for the lower legs of a user in a prone position with the midsection of the user on said pelvis pad.

26. A therapy device as in claim 25, wherein said shin pad is substantially cylindrical in shape.

27. A therapy device as in claim 1, further comprising a pad cover located over said pliable pad, the pad cover also including an elastic section disposed adjacent to said bladder.

28. A therapy device for providing lumbar manipulation comprising:
a substantially rigid frame;
a pliable pad with a cavity, the pad supported on said substantially rigid frame;
a bladder supported on said substantially rigid frame and received by said cavity; and
a pump in fluid communication with said bladder, whereby actuation of said pump enables expansion of said bladder providing vertical displacement of a portion of said bladder relative to said substantially rigid frame.

29. A therapy device as in claim 28, wherein said at least one cavity is substantially cylindrical in shape.

30. A therapy device as in claim 28, wherein said support frame is comprised substantially of a channel with a pair of sloping legs.

31. A therapy device as in claim 30, wherein said channel includes a substantially hollow underside.

32. A therapy device as in claim 28, wherein said bladder is a collapsible bladder.

33. A therapy device as in claim 28, wherein said bladder is comprised of two substantially cylindrical shaped bladders made of a pliable material.

34. A therapy device as in claim 33, wherein said two substantially cylindrical shaped bladders are positioned eleven inches apart.

35. A therapy device as in claim 28, wherein said pump is an electrically powered air pump.

36. A therapy device as in claim 28, wherein said pump is operated by use of a switch remotely positioned from said pump.

37. A therapy device as in claim 28, wherein said pump is hand actuated.

38. A therapy device as in claim 28, further comprising a substantially U-shaped face pad with an open center section and an upper connecting section.

39. A therapy device as in claim 28, further comprising a pliable shin pad, whereby said shin pad provides support for the lower legs of a user in a prone position with the midsection of the user on said pelvis pad.

40. A therapy device as in claim 28, further comprising a pad cover located over said pliable pad, the pad cover also including an elastic section disposed adjacent to said bladder.

41. For use with a therapy device including a frame, a pliable pad with a cavity, the pad supported on said frame, a bladder supported on said frame and received by said cavity, and a pump in fluid communication with said bladder enabling expansion of said bladder, a method of spinal manipulation including the steps of:
positioning a user in a prone position with the user's illiac crest over said bladder;
actuating said pump, thereby expanding said bladder, displacing an upper portion from said frame; and
providing posterior rotation of the user's pelvis by a force applied to said illiac crest from said upper portion of said expanding bladder.
